(19) United States
(12) Patent Application Publication
(10) Pub. No.: US 2026/0055465 A1
(43) Pub. Date: Feb. 26, 2026

(54) Novel markers for detection, isolation and targeting of circulating tumor cells

(71) Applicant: Stefano Volinia, Ferrara (IT)

(72) Inventor: Stefano Volinia, Ferrara (IT)

(21) Appl. No.: 18/811,711

(22) Filed: Aug. 21, 2024

(51) Int. Cl. C12Q 1/6886

(52) U.S. Cl. CPC C12Q 1/6886; C12Q 2600/118; C12Q 2600/158

(57) ABSTRACT

This application describes methods for purification and subtyping of circulating tumor cells (CTCs), cells that are shed by the tumors into blood. Methods include analyzing functional modules shared by CTC and trophectoderm, methods for determining the likelihood of the primary tumors to metastasize, and methods of treating a cancer determined to be likely to metastasize.

### CROSS-REFERENCE TO RELATED APPLICATIONS

This application claims priority to U.S. Provisional Application No. 63/534,052 filed on Aug. 22, 2023, the entire contents of which are incorporated by reference.

### BACKGROUND OF THE INVENTION

Metastasis is the major culprit in the deadly role of cancer, with estimates ranging to 66% to 90% of cancer deaths due to metastasis¹.

Metastasis can be formulated as a multistep process that unravels through the dissemination of circulating tumor cells (CTCs) by the tumor and the colonization of distant organs², with the formation of metastasis³. Higher number of CTCs detected in cancer patients is associated with shorter survival.

Because CTCs are of epithelial origin their fitness in circulation is thought to be diminished, due to phenomena such as anoikis, turbulence, and the action of the immune system. Therefore, disclosing the modalities that CTCs employ in this environment could lead to the design of novel therapeutic strategies. To overcome their struggle in the circulation, CTCs can partner, physically or functionally, with platelets, erythrocytes, neutrophils, macrophages, natural killer (NK) cells, lymphocytes, endothelial cells, and cancer-associated fibroblasts^{4,5}. Interestingly, it was recently shown that CTC intravasation mostly occurs during sleep⁶.

Cancer cells can exist in different states within the tumors, and are associated with distinct functions, such as proliferation, differentiation, invasion, metastasis, and resistance to drugs⁷. These various tumor cell states are being dissected by single-cell NGS studies^{17,8}, thus exposing the pathways which promote and maintain these functional states and regulate their transitions^{199,10}. The tumor microenvironment is further integrated by the apport of stromal cells¹¹.

Many clinical and molecular investigations have established branched evolution as a substantial feature of cancer¹². A Darwinian model has been sustained by the sedimentation of somatic changes generated through many small and some large cohort studies^{13,14} (TCGA, COSMIC). Metastasis is the result of cellular processes which span invasion, migration and epithelial-mesenchymal transition, and have relations with the tumor microenvironment¹⁵. Key components of these processes include the TGFβ-ZEB1/ZEB2 axis and the nuclear factor-κB pathway and can also be regulated by microRNAs or other noncoding RNAs¹⁶. Investigating disseminated tumor cells in bone marrow and CTCs in peripheral blood has been an important source of knowledge on cancer progression and metastasis^{17,18}. Currently, single cell RNAseq (scRNA-seq) and DNA-seq represent mature technologies to help unravel the molecular evolution of cancer in patients. Unfortunately, CTC studies have been limited by the availability of specific markers for their purification from blood.

There is a need to enable comprehensive harvesting of CTC from patients, extending the range of CTC purification platforms and furthermore there is a need to predict cancer progression and metastasis using either solid or liquid biopsies. To this purpose, 3302 RNAseq profiles of samples labeled as CTCs in the GEO or SRA databases¹⁹ were collected and integrated. Most of the CTCs analyzed hitherto originated from patients with breast cancer, but CTCs from patients with cancer in other organs were also included (e.g., lung, prostate, stomach, colon, pancreas, and liver).

### SUMMARY OF THE INVENTION

Disclosed herein are methods for analyzing the metastatic potential of circulating tumor cells (CTCs), and of primary tumors, in the context of breast, prostate, lung, stomach, pancreas and colon cancer.

In embodiments, the disclosure provides a method for analyzing the functional modules shared between CTCs and embryonic cell types, like in particular the trophectoderm, where the method includes defining at least one key feature of the CTC/trophectoderm modules in a biological sample from a subject, and assessing the role of the at least one key feature of CTC/trophectoderm modules during tumor progression. In some embodiments, the at least one key feature is a transcriptome, or a proteome. In some embodiments, activation of CTC/trophectoderm modules includes a presence of circulating tumor cells (CTCs). In some embodiments, the presence of CTCs with activated CTC/trophectoderm modules indicates an increased likelihood of metastases. In embodiments, a research tool for predicting tumor metastases is provided, where the tool utilizes a method for analyzing CTC/trophectoderm modules. In some embodiments, the method includes defining at least one key feature of a CTC/trophectoderm module, and assessing the role of the at least one key feature of the CTC/trophectoderm module during tumor progression.

In embodiments, a method for determining the likelihood of cancer metastases is provided, where the method includes performing RNA sequencing on a biological sample from a subject to identify expressed genes of the CTC/trophectoderm modules, determining an expression composite score for each of the expressed genes, and using the composite score to assess the activation of the CTC/trophectoderm modules, where cancer cells with activated CTC/trophectoderm modules are more likely to metastasize. In some embodiments, activated CTC/trophectoderm modules includes a presence of circulating tumor cells (CTCs). In some embodiments, the presence of CTCs indicates an increased likelihood of metastases.

In embodiments, the disclosure provides that the methods further comprise methods of treatment of a cancer comprising determining the likelihood of tumor metastases as described herein, and administering to a subject determined to have an increased likelihood of tumor metastasis an effective amount of a treatment for increased metastasis of the cancer as known in the art. The effective amount to be administered is determined based on the type of cancer and the degree of determined increase in likelihood of tumor metastasis as described herein.

### BRIEF DESCRIPTION OF DRAWINGS

FIG. 1. Aneuploid cells as inferred by RNAseq profiles using copyKat; cells with copy number variations (CNV) are indicated as red dots (left panel).

Aneuploid cells as inferred by RNA profiles using copyKat (right panel; cells with CNVs are blue).

FIG. 2. Cell cycle analysis of candidate CTCs (Harmony integration). Tricycle designs 0.5pi to be the start of S stage, pi to be the start of G2M stage, 1.5pi to be the middle of M stage, and #. 1.75pi-0.25pi to be G1/G0 stage.

FIG. 3. Cell cycle analysis of aneuploid bona fide CTCs (circle Kernel density plot). Aneuploid cells (bona fide CTCs) appear as mostly in G2/M, while most of the diploid cells are in G1/Go phase.

FIG. 4. Scanorama integration of QC-passed CTC and PBMC single cell datasets.

FIG. 5. Scanorama integration of QC-passed CTC and PBMC single cell datasets (epithelial marker EPCAM, mesenchymal marker CAV1, prostate specific marker KLK2, and KRT8).

FIG. 6. Violin plots with the expression of CTC and PBMC markers upon separation of bona-fide CTC based on aneuploidy.

FIG. 7. the cell cycle in the CTC subgroups and PBMC.

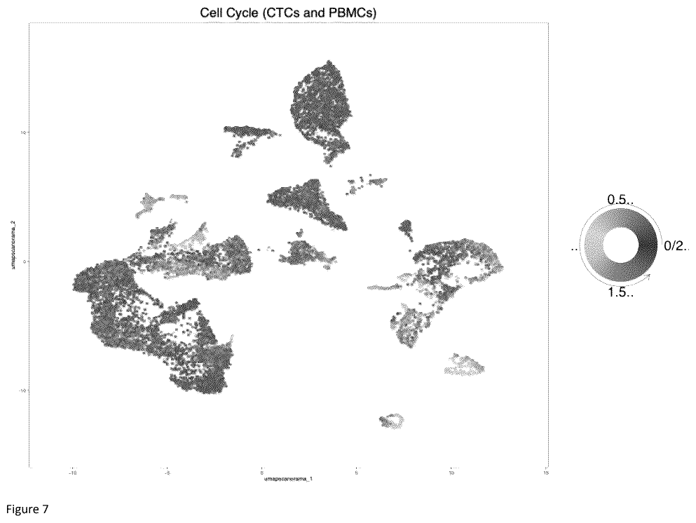

FIG. 8. The expression of classical CTC markers in the three bona-fide CTC subgroups: Epithelial A), epithelial B and mesenchymal. Albeit VIM is diagnostic for mesenchymal subtype within CTCs, it is also expressed in PBMCs and therefore it is not strictly CTC-specific.

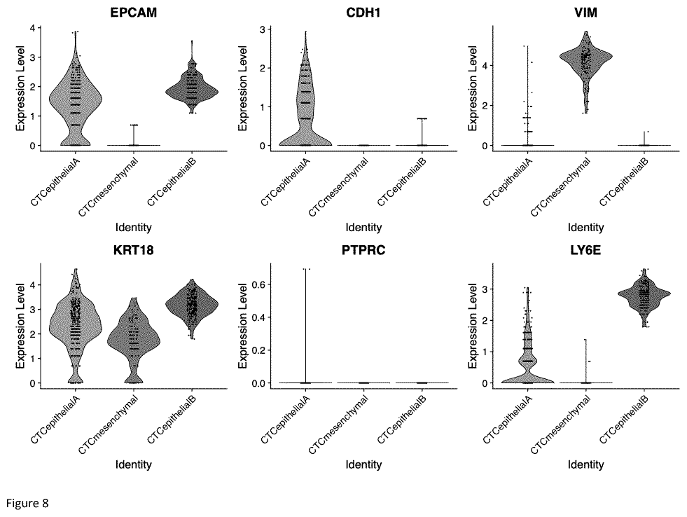

FIG. 9. Scanorama integration of the CTC and PBMC sca 10K datasets. A) CTC and PBMC 10K datasets: UMAP of the Scanorama integration. The PBMC (violet) used as reference for blood cells occupy the center and left portions of the UMAP. The aneuploid bona fide CTCs (red) and diploid candidate CTC (green) are located in the bottom and righthand side. The non-cancer contaminant cells (azure) that co-purify along with CTCs are plotted in the right and central portion of the figure. B) The three bona-fide CTC subgroups: Epithelial A (red), epithelial B (olive) and mesenchymal (green) are in the right bottom quadrant, under the diploid contaminant (non-CTC, azure). the PBMC clusters (pink) are in the center and left portion of the UMAP.

FIG. 10. Differentially expressed genes in epithelial and mesenchymal CTCs subdivided by tumor of origin. The genes with highest ROC AUC in each group were selected and non-membrane markers were removed. AXL, EMP1, CAV1 and NT5E are mesenchymal specific markers. LY6E is specific for Epithelial B CTCs.

FIG. 11. The kernel distribution of cell cycle in the three CTC subgroups. The mesenchymal and the epithelial B CTCs are the most engaged in the cell cycle, while the epithelial A CTC are mostly in G0/G1 phase.

FIG. 12 Immune-checkpoint targets in CTCs. Expression of PDL1 (CD274) and of B7-H3 (CD276), two clinically relevant targets for immune-therapy.

FIG. 13. The markers indentified in our work, include several novel markers, both for epithelial or mesenchymal CTCs. To identify robust markers, we used 10-fold nested cross validation and glmnet. A pan-CTC model (including all CTCs, irrespective of their epithelial or mesenchymal status) was generated by comparing all true CTCs with the negative controls, including the PBMCs (n=10,434) from the Broad SCA dataset and the contaminant cells isolated from blood using microfluidics (n=1,582).

FIG. 14. UMAP of the CTC, cancer and embryonic cell types upon Scanorama integration

FIG. 15. Divisive hierarchical spectral clustering of CTCs, metastatic and non-metastatic primary tumors, metastatic lymph nodes and trophoblasts from normal placenta²⁰. Smart tree pruning (K=0.5) of spectral clustering visualizes single cell profiles, while providing weighted average blending of colors and scaling branches. The input expression matrix was shifted log transformed prior to clustering.

FIG. 16. CTCs map onto the trophectderm (TE) space in the embryo reference map (left panel). The CTCs (n=72) had similarities with trophectoderm (TE), the layer of cells in the blastocyst, which gives rise to the cell in the trophoblast and allows the attachment of the embryo to the endometrium and its successive invasion to form the placenta (positive sample are red dots in the second panel). No cells with TE-like phenotype were present in normal breast, nor in primary tumors from breast cancers (third panel). The trophoblasts cells (CTB, STB, and EVT) were correctly mapped onto the reference UMAP plot (last panel). Cells from metastatic lymph nodes and the corresponding breast tumors have only 1 TE-like sample (not shown

FIG. 17. The UCell composite scores with the CTC/trophectoderm RNA modules, co-regulated in CTCs, metastatic breast cancer and embryonic cells, were computed and the results for the key modules are shown on the breast cancer-CTC-embryo integrated UMAP plots

FIG. 18. The UCell composite scores with the CTC/trophectoderm RNA modules, co-regulated in CTCs and trophoblasts, were computed and the results for the key modules are shown on the breast cancer-CTC-embryo integrated UMAP plots

FIG. 19. The CTC RNA modules, up-regulated only in CTCs, with their composition.

FIG. 20. Markers for removal of PBMCs and blood contaminants from CTCs (after size-based microfluidics enrichment). The expression of the markers in the two models (PBMC and contaminants vs bona fide CTC, boxes 2, 3) or contaminants vs other samples (PBMC and CTCs, box 1) are visualized in the dotplot. Some CTC markers and reference genes are also included (boxes 4, 5, 6).

### DETAILED DESCRIPTION OF THE INVENTION

Various further aspects and embodiments of the disclosure are provided by the following description. Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M.J. Gait, ed., 1984); Animal Cell Culture (R.I. Freshney, ed., 1987); Methods in Enzymology (Academic Press, Inc.); Current Protocols in Molecular Biology (F.M. Ausubel et al., eds., 1987, and periodic updates); PCR: The Polymerase Chain Reaction (Mullis et al., eds., 1994);

Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a cell, a pharmaceutical composition, and/or a method that “comprises” a list of elements (e.g., components, features, or steps) is not necessarily limited to only those elements (or components or steps), but may include other elements (or components or steps) not expressly listed or inherent to the cell, pharmaceutical composition and/or method.

As used herein, the transitional phrases “consists of' and “consisting of' exclude any element, step, or component not specified. For example, “consists of' or “consisting of' used in a claim would limit the claim to the components, materials or steps specifically recited in the claim except for impurities ordinarily associated therewith (i.e., impurities within a given component). When the phrase “consists of' or “consisting of' appears in a clause of the body of a claim, rather than immediately following the preamble, the phrase “consists of' or “consisting of' limits only the elements (or components or steps) set forth in that clause; other elements (or components) are not excluded from the claim as a whole.

As used herein, the transitional phrases “consists essentially of' and “consisting essentially of' are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of' occupies a middle ground between “comprising” and “consisting of'.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of′ aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

In an aspect, the disclosure provides a method of treating or preventing tumor metastases in a subject in need thereof, comprising administering an effective amount of a treatment for the tumor and/or tumor metastases to the subject. In some embodiments, the cancer is breast cancer.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that that can be treated by any means known or discovered in the future. Suitable subjects (e g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

A model system for studying propensity, particularly in circulating tumor cells, for initiating breast cancer metastases, is provided. This model may include evaluating circulating tumor cell subtype by defining the metastatic and CTC/trophectoderm modules in transcriptome, proteome, and phenome, and unravel their role during cancer progression.

We identified three different CTC subgroups: epithelial A (CDH1+/EPCAM+), epithelial B (CDH1−/EPCAM+), and mesenchymal (VIM+).

Novel CTC markers were determined spanning all subgroups^{21,22}, specific for epithelial CTCs, or for mesenchymal CTCs such as AXL and CAV1.

We unveiled that CTCs share RNA modules with the cells from the trophectoderm layer in the early embryo. This functional convergence might be at the basis of the invasiveness capabilities that CTCs share with trophectoderm.

We also unveiled that primary tumors from patients with metastatic lymph nodes share genetic programs with the CTCs.

We determined a number of novel markers (unrelated to other known markers, such EPCAM, keratins, or ERBB2) to isolate CTCs from blood samples using immunepurification.

As applied to clinical use, RNA sequencing and/or gene extraction may be beneficial in determining the subclass and the metastatic potential of the CTCs present in patients'blood. This may be achieved by extracting RNA from blood, however this may be difficult due to the limited quantity of cells in blood samples and/or the efficiency of RNA extraction from blood samples, so alternative methods for determining metastatic potential of CTCs may be required. For example, in some embodiments, the gene signature for the metastatic potential of CTCs in a patient is identified using microfluidics to enrich large CTC from blood. In other embodiments, single cell RNA sequencing technology is used to determine the metastatic potential of CTCs.

As again applied to clinical use, RNA sequencing and/or gene extraction may be beneficial in determining the metastatic potential of the primary tumors in a patient. This may be achieved by single cell RNA sequencing technology.

### EXAMPLES

### Study Design

Twenty-seven datasets containing RNA profiles from samples annotated as CTCs were obtained from GEO and SRA (GSE104209, GSE109761, GSE111065, GSE111842, GSE113890, GSE114704, GSE115501, GSE117623, GSE126669, GSE129474, GSE144494, GSE144495, GSE144561, GSE180097, GSE186288, GSE198291, GSE208448, GSE51827, GSE67939, GSE67980, GSE74639, GSE75367, GSE86978, PRJDB11367, PRJNA471754, SRP281893, SRP335264) and processed to yield a merged expression matrix with 3302 CTCs, called here putative CTCs. Notably, these RNA profiles were generated using two routes, either by scRNAseq or by RNAseq of single purified cells. A gene was removed from the expression matrix when its counts were not determined for more than 5% of the samples, otherwise undetermined counts were imputed as the median expression for that gene. Heterogeneous CTC samples, likely to be doublets or multiplets, were identified and removed using scDblFinder²³. Additional quality control filtering was performed to remove samples with high percentage of gene expression for hemoglobin (>25%), or for platelet markers (>5%). The raw counts in the merged expression matrix were then transformed and variance-stabilized using the shifted log method²⁴. Harmony, alongside Scanorama, FastMNN, scVI, LIGER and Seurat (CCA or RPCA) were used for integration analysis²⁵. Harmony was chosen to integrate the project (candidate CTC datasets), while Scanorama was used to integrate the larger projects, including PBMC or cancer scRNAseq datasets. A resolution of 2.5 was used to detect the clusters with the Louvain algorithm. Azimuth, scATOMIC²⁶ and scLearn²⁷, were applied to the automatic cell-type identification of contaminating peripheral blood mononuclear cells (PBMCs) in the putative CTCs. In orthogonal fashion, the determination of chromosomal aneuploidy for the CTCs, was performed using both copyKATand scevan. The complex and highly heterogeneous single cell profiles from tumors and from lymph nodes, went through classification and purification stages. For example, we removed blood derived cells (i.e., those in clusters showing high CD45/PTPRC, CD52, CXCR4 or CD74 expression) from cancer tissues, while endothelial cells were identified for positivity to SELP or VWF). Tricycle was employed to infer and visualize cell cycle processes. The Human Protein Atlas supported cell type specific expression and subcellular protein localization. We used the Wilcoxon test coupled with ROC and AUC for the selection of CTC markers. The Broad Institute PBMC Systematic Comparative Analysis dataset²⁸ (pbmcsca) was the reference for PBMCs in the integration with CTCs. For the placenta and for the breast normal and cancer tissues, we obtained single cell RNA-seq data from the GSE89497/PRJNA352390 and the GSE161529 (GEO studies, respectively.

Additionally, for single cell profiling of primary and paired metastatic lymph node tumors in breast cancer patients, GSE167036. Finally more normal breast profiles were obtained from the HBCA project. For the PRJNA352390, raw counts were obtained from the fastq files using STAR. The clustree R package was applied to choose the optimal resolution for clustering of the integrated CTC/breast/trophoblast datasets. Gprofiler was used for functional annotation of gene lists. Finally, nested 10-fold cross-validation with elastic-net regularised linear models and caret were used to derive robust models for CTCs in PBMC background. R, Bioconductor and RStudio (Posit Software, PBC, Boston, MA, USA) were used for the scRNA-seq analysis described above, alongside Python.

### The CTC Single Cell Profiles Integrated From Public Databases Contain a Large Fraction of Contaminating Blood Cells

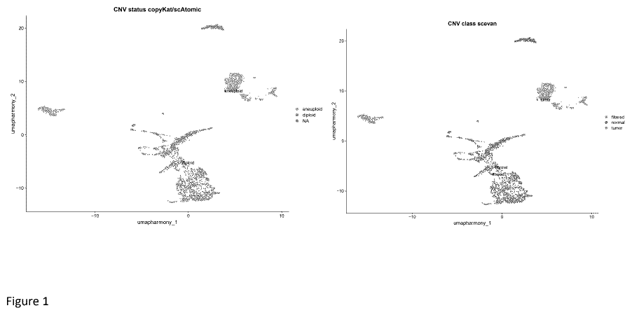

The transcriptomes of 3302 putative CTCs were studied (27 datasets from GEO or SRA public databases). Most of the CTCs were derived from blood of patients with breast cancer, but patients with pancreas, prostate, lung, liver, stomach and colorectal cancers, or melanoma were also represented. The raw counts for 12,987 genes were transformed, and variance stabilized, using shifted log. The datasets were integrated using Harmony and Seurat. Our first goal was to confirm the identities of these putative CTCs, e.g., whether they were epithelial or mesenchymal CTCs, cancer or even non-cancer cells. For this purpose, we conducted an exploratory analysis by plotting the expression levels of known CTC markers (like EPCAM, KRT8, KRT18, VIM), or PBMCs (PTPRC/CD45) as violin plots over the Harmony clusters. Several Harmony clusters had cells positive for KRT8 and KRT18, EPCAM or VIM, and negative for PTPRC/CD45, representing thus candidates for bona-fide CTCs. The remaining clusters had expression of markers for platelets (PPBP) and were negative for all CTC markers. Notably, while EPCAM was restricted to a subset of epithelial (KRT8 /KRT18 positive) cells, VIM expression was shared by KRT+ and KRT− negative clusters. From this coarse, but effective, exploratory analysis of the integrated datasets, it emerged that a large portion of the putative CTCs were likely non-cancer cells. We needed to further characterize the samples, and ultimately separate any bona-fide CTC from contaminating non-cancer blood cell. We decided to measure chromosomal rearrangements for the identification of cancer cells. Cancer cells, like CTCs, often bear multiple and extensive copy number variations (CNVs) in their chromosomal repertoire (aneuploidy), which are absent from non-cancer cells. Therefore, we applied different CNV tools, scATOMIC, copyKAT and scevan, to infer aneuploidy from RNA profiles. Aneuploid cells were plotted as red dots in FIG. 1A (copyKAT) and as azure dots in FIG. 1B (scevan). Reassuringly, with the only exception of the diploid cluster #8, in the Harmony UMAP plot there was an almost complete visual overlap between aneuploid cells (highly likely to be cancer cells) and those positive for KRT8 /KRT18 (cells of epithelial origin).

At this stage we had labeled CTCs in two ways: a) as cells which belonged to clusters expressing the classical CTC markers (KRT+/EPCAM+/CD45−, or KRT+/VIM+/CD45−) or b) as aneuploid cells. These two parameters led to the selection of candidate CTCs (and conversely also of putative blood cell contaminants). Nonetheless, we were aiming to identify a highly purified cohort of CTC cells, and it is well known that circulating tumor cells often complex with non-cancer cells or can be found in multicellular groups. Thus, with the goal of performing a stringent selection towards a true single cell CTC dataset, we manually curated each candidate CTC sample according to its annotation, deposited in the public databases. The curation allowed us to remove 224 samples which were not annotated as single cells (e.g., CTC clusters), or which were briefly in-vitro cultured. This manual curation of selected CTCs generated a cohort of 872 candidate single CTC profiles (discarding almost ¾ of the original CTC-labelled samples).

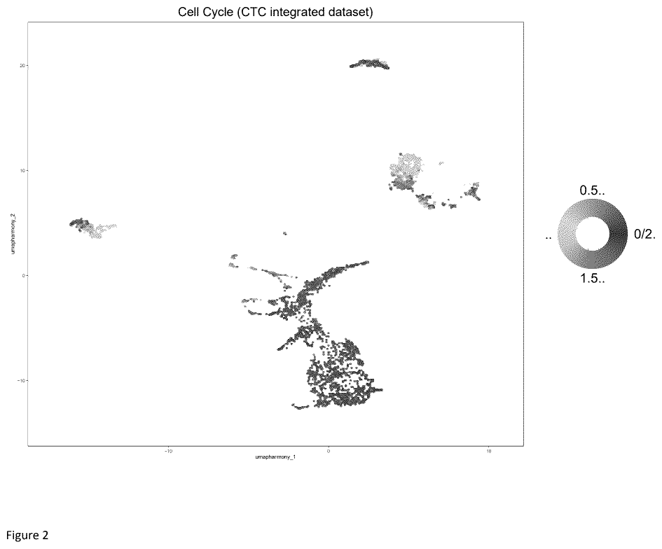

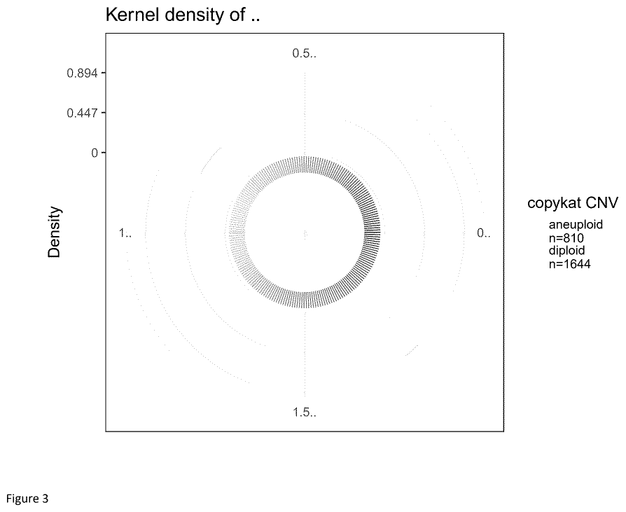

We then studied the cell cycle in the integrated CTC dataset using Tricycle. As indicated in the UMAP and in the circle kernel density plots (FIGS. 2 and 3, respectively), most of the cells in S and G2/M phases (pink to yellow to light green dots) were also aneuploid, while most of the diploid cells were in G1/G0 phase (blue dots). These findings were well in agreement with what one would expect for cancer cells and blood-derived contaminant cells, respectively.

### Integrative Analysis of CTCs and PBMCs Identifies Three Subgroups of Bona-Fide CTCs

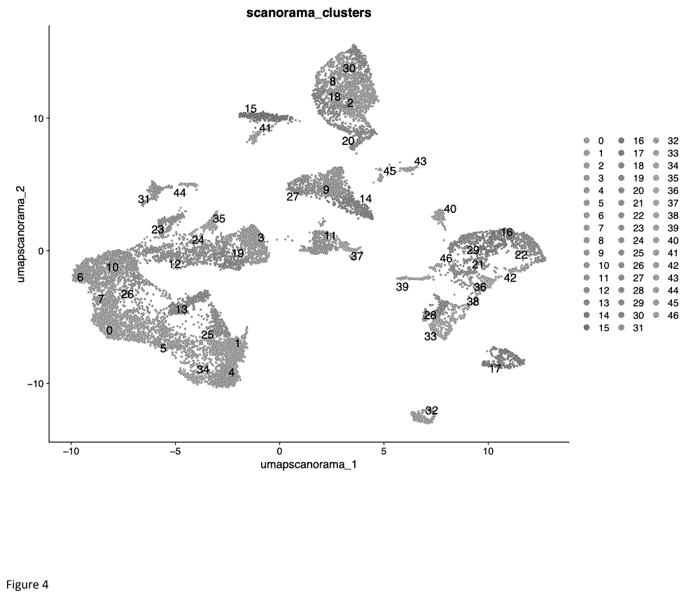

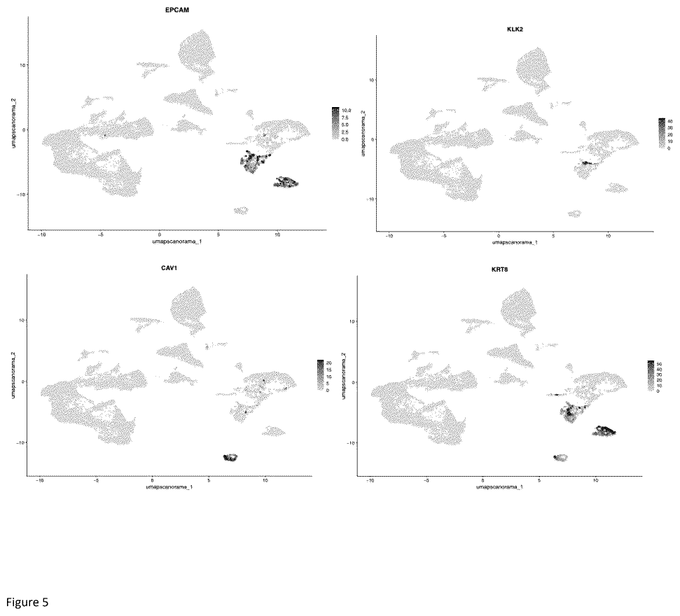

Since the non-cancer, or contaminant, cells were blood-borne, to recognize them we integrated the CTC datasets with scRNAseq profiles derived from peripheral blood mononuclear cells (PBMCs, n=10,434). In particular, we used the Broad Institute PBMC Systematic Comparative Analysis dataset. The unintegrated project had seven KRT8 /KRT18 positive clusters out of 57 clusters; the integration with Scanorama had five KRT+ clusters out of 47 in total, in comparison with five out of 32 with FastMNN and eight out of 36 with Harmony. FastMNN was the most aggressive in cluster reduction, while Scanorama led to the lowest number of CTC clusters without over-integration of the PBMC datasets. Thus, we went on to tackle the cellular complexity by overlaying the Scanorama cluster map (FIG. 4) with the PBMC and CTC annotations (e.g., aneuploid or diploid CTCs, FIG. 5). The largest group of clusters on the left and center of the Scanorama UMAP was from PBMCs (#0-15, 18-20, 23-27, 30, 31, 34, 35, 37, 41, 43-45). The group of clusters on the right side contained the bona-fide CTCs (#17, 28, 32, 33, 38) and the putative diploid blood contaminants (#16, 21, 22, 29, 36, 39, 40, 42, 46), as determined in the earlier analysis steps, prior to the integration with PBMCs.

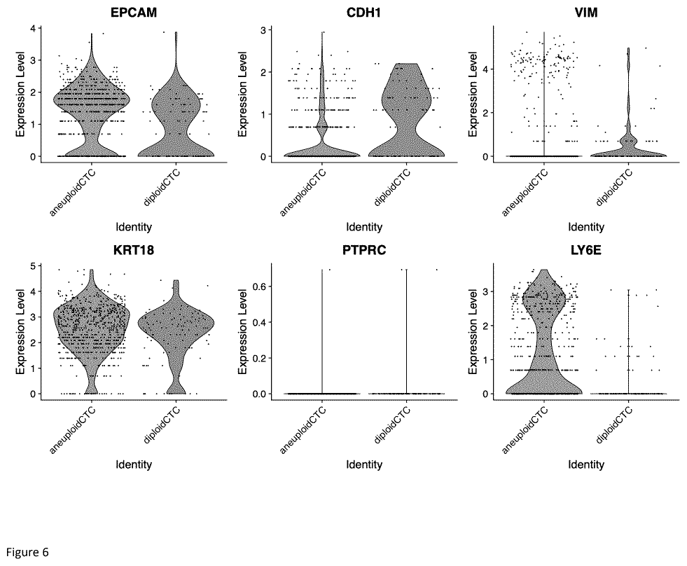

Furthermore, feature plots (FIG. 5) visualize the expression of CTC markers in each sample over the Scanorama integration cluster map. The KRT8/18+ clusters were in the right and bottom portion of the UMAP and as expected, largely overlapped with the aneuploid CTCs (FIG. 6). As a confirmation, CTCs from patients with prostate cancer were correctly placed in the bona-fide CTC clusters and overlapped with the expression of the prostate specific KLK2 gene (FIG. 5). Further corroborating our CTC selection procedure, none of these bona-fide CTC clusters contained cells from the PBMC dataset. FIG. 6 show the expression of CTC and PBMC markers in the bona-fide CTC clusters and upon CTC stratification by inferred ploidy.

Having identified blood-derived contaminants among the CTC datasets, we then proceeded with the accurate reassessment of each CTC. Among the aneuploid CTCs, only 9 samples (1.4 %) were located outside the CTC clusters and were therefore discarded from subsequent analysis. The scarcity of aneuploid CTCs mapping outside the KRT+ CTC clusters supported the validity of using aneuploidy as a major criterium, albeit non-permissive or too stringent, for CTC identification. Nonetheless, a large fraction of the cells in the KRT+ CTC clusters (about 40%) were still predicted as diploid. For this reason, we decided to retain as bona-fide CTCs those candidate CTCs called from the Harmony integration in stage I, that were still belonging to the CTC clusters in the Scanorama integration (with the PBMCs) in stage II.

A source of uncertainty, which we wished to address, was in the experimental identification of CTCs, generally performed using immunophenotyping. Therefore, to reinforce our stringent CTC selection we run automated cell type annotation on the cohort of bona-fide CTCs. Using scLearn we used mammary gland and hematopoietic tissues, bone marrow and PBMC, while with Azimuth, we could leverage the references for PBMCs and for fetal tissues. Eighty-three CTC samples (with large excess among the diploids) were predicted as platelets and were removed leaving a final dataset of 731 bona-fide, single cell and pure CTCs. Some residual expression of platelet specific genes (i.e. PPBP or PF4) was still present in less than 10% of the bona-fide CTC samples but was essentially compatible with the reported coating of CTCs by platelets. The cell cycle analysis was performed on the integrated CTC/PBMC dataset and confirmed that the aneuploid CTCs were engaged in the S phase while the diploid cells in the CTC clusters, most of the contaminant cells and of PBMCs were not.

At this stage it was relevant to underscore that the integrated bona-fide CTC dataset we obtained had a very skewed representation of the different cancer types, and that CTCs from breast cancer (n=624, i.e. 85.3%) were by far the most frequent, followed from those from prostate (n=88).

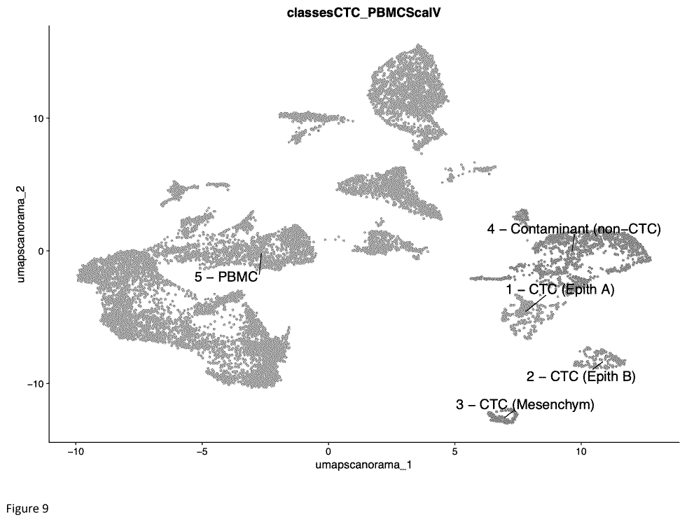

One of our major goals was to identify novel and informative markers for the detection, purification, or targeting of the rare CTCs. Upon integration, we had classified such CTCs in two ways: 1) as cell expressing the classical CTC markers (KRT+/EPCAM+/CD45−, or KRT+/VIM+/CD45−) or 2) as aneuploid cells harboring copy number variations. These two orthogonal features led to a first selection of strong candidate CTCs (and conversely also of putative blood cell contaminants). The bona-fide CTCs could be stratified into three subpopulations, as determined by the Scanorama UMAP (FIG. 4) and by the expression of CTC classic markers (FIG. 5, and FIG. 9): the EPCAM+ epithelial A cells were CD24+ and CDH1+, while epithelial B and mesenchymal were not. Among novel markers, VIM, CAV1 and AXL genes showed the highest specificity for the mesenchymal cluster (AUC ROC>0.99), while IDH2 and LY6E were the most diagnostic for epithelial B CTCs. Notably, the last two genes are also highly expressed in trophoblast tissues (as shown at Human Protein Atlas web site).

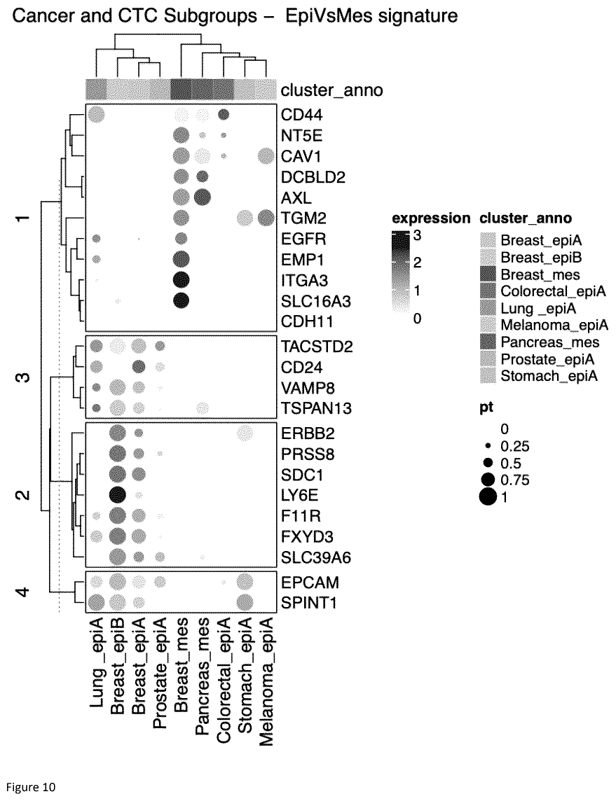

FIG. 10 shows the diagnostic genes in each of the CTC subgroups and by cancer of origin.

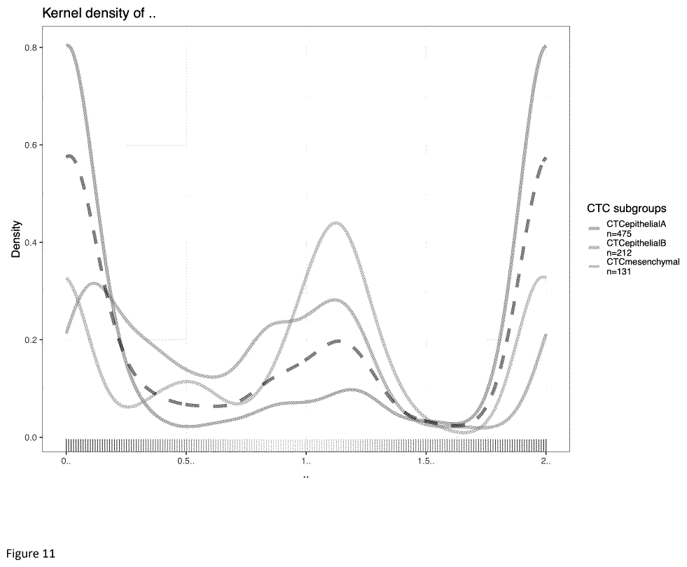

One key information with clinical importance can now be obtained: FIG. 11 shows the kernel distribution of cell cycle in the three CTC subtypes. The mesenchymal and the epithelial B CTCs are the most engaged in the cell cycle, while the epithelial A CTC are mostly in G0/G1 phase. This difference could be very important towards the patient prognosis.

### Clinically Relevant Markers: Targets for Cancer Immunotherapy of CTCS

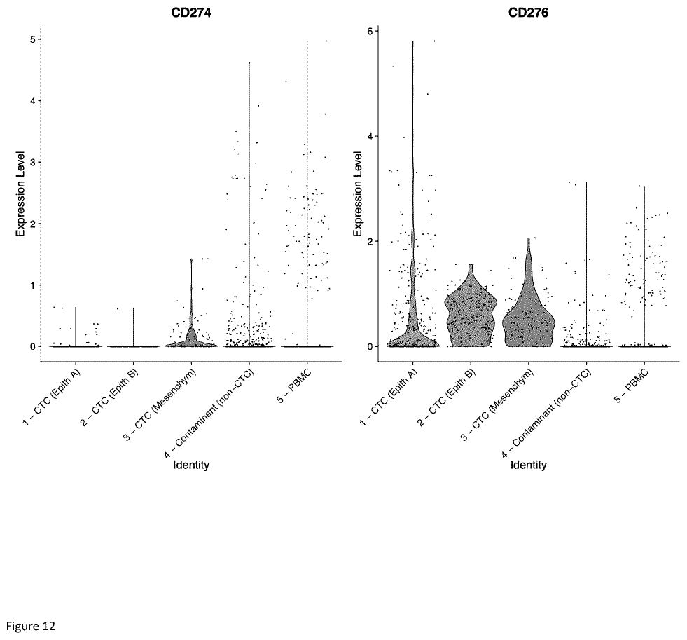

The availability of a CTC/PBMC integrated single cell dataset gave us the unique opportunity to explore some clinically relevant markers. We started by looking at the CD markers and those which are differentially expressed in the various cancer and CTC subgroups and in the controls. CD24 was highly expressed in Epithelial A breast CTCs. PDL-1 (CD274) that represents a clinical target for immunotherapy, was expressed only in mesenchymal breast CTCs and at a very low level, but not in the other CTCs. Nonetheless, when we explored the RNA levels of several other genes involved in immune checkpoint, two of them were expressed in breast and prostate CTCs: CD276 (B7-H3, FIG. 12), and PVR (CD155). Both genes are involved in immune-suppression and appear here as coding for two possible targets in immune-therapy of circulating tumor cells. CD276, like PDL-1, participates in the regulation of T-cell-mediated immune response and may play a protective role in tumor cells by inhibiting natural-killer (NK) mediated cell lysis. Very interestingly CD276 is also highly expressed in trophoblast and at higher levels than PDL1. Additionally, other CD proteins overexpressed in CTCs hint at dampening of the immune activation: for example, CD46, has cofactor activity for inactivation of complement components C3b and C4b by serum factor I, which protects the host cell from damage by complement. CD59 is a potent inhibitor of the complement membrane attack complex (MAC) action. CD9 might prevent the macrophage fusion into multinucleated giant cells specialized in ingesting complement-opsonized large particles. CD70 and CD163L1 are restricted to mesenchymal CTCs and might regulate the CTC crosstalk with immune components as well. CD63 instead plays a role in the adhesion of leukocytes onto endothelial cells via its role in the regulation of SELP trafficking, and thus can help CTC to bind to endothelia.

### Novel and Informative Markers for Identification of CTCS From Blood

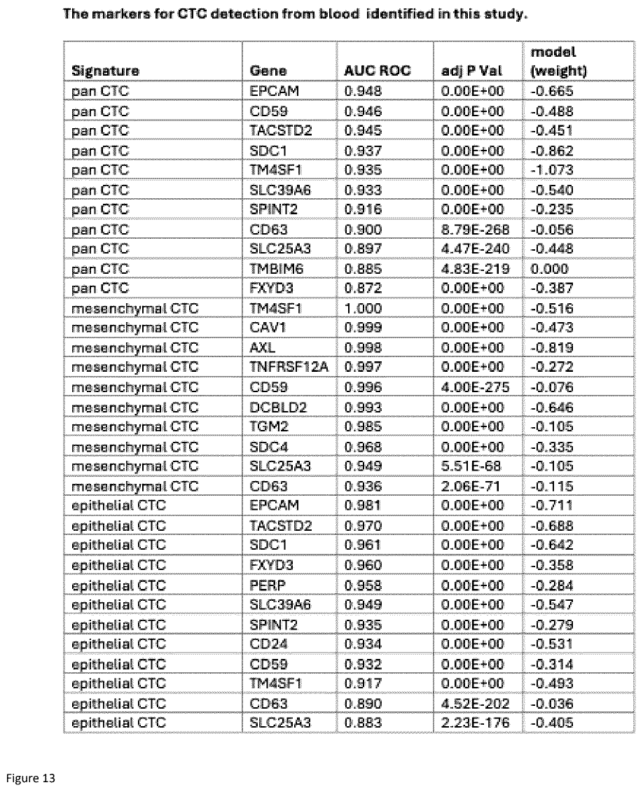

What are the best markers to isolate CTCs from PBMCs and other non-CTC contaminants found in blood? At first, we looked for pan-CTC markers, i.e., those markers capable of selecting CTCs, irrespective of their status (epithelial or mesenchymal), or tissue of origin. Before running the analysis, since the CTCs were mitotically active when compared with PBMCs and contaminant cells, we removed from the analysis all genes involved in cell cycle and proliferation, as well as ribosomal genes. To identify robust markers, we used 10-fold nested cross validation and glmnet. A pan-CTC model (including all CTCs, irrespective of their epithelial or mesenchymal status) was generated by comparing all true CTCs with the negative controls, including the PBMCs (n=10,434) from the Broad SCA dataset and the contaminant cells isolated from blood using microfluidics (n=1,582). Because we aimed to the identification and purification of CTCs from blood using antibodies, we focused only on genes encoding membrane proteins (n=3085). The results for all CTCs, and for the epithelial or mesenchymal subgroups, are summarized in FIG. 13. All three models showed very high balanced accuracies (above 0.98). The role of EPCAM was confirmed in the epithelial CTC model, where it was the most informative gene, closely followed by TACSTD2. Nevertheless, since EPCAM-directed antibodies were used to isolate most of the CTCs in our collection, this finding was not surprizing and might not reflect the real frequency of EPCAM positive CTC in the blood of patients with cancer. As expected, EPCAM was not included in the mesenchymal CTC model, where several novel CTC markers appeared, such as AXL, DCBLD2, TM4SF1, CAV1, SDC4, TNFRSF12A, CD63, TGM2, SLC25A3 and CD59.

### Circulating Tumor Cells Share RNA Profiles With Cells From the Early Embryo

Is there a relation between CTCs and primary tumors? We do not have here the single cell profiles for the exact same tumors from which the CTC originated, but we do have a large amount of single cell profiles from the various subtypes of breast cancers, from patients with or without metastasis. Furthermore, is there a relation between CTCs and trophoblast cells, as hinted by some highly expressed genes, such as for example CD276, IDH2 and LY6E, that are in common between the two cell types?

To answer these two fundamental questions, we proceeded to study the possible connections of CTCs with cancer cells from solid tumors and placenta tissues. Since most of the bona-fide CTCs (85.3%) were from patients with breast cancer, we integrated the CTC single cell profiles with those from normal breast and from the different breast cancer subtypes: ER+, HER2+, TNBC. Additionally, because CTCs are implicated in metastasis, we also integrated single cell profiles from metastatic lymph nodes and their respective primary tumors (here of ER+ subtype). Finally, we also integrated cells early embryos and from first-and second-trimester human placentas, including cytotrophoblast cells (CTBs) and extra-villous trophoblast cells (EVTs). To avoid influences due to mitotic activity, which might be present in a subset of the cells, we removed the cell cycle genes (n=97, Seurat S and G2 genes).

In addition to the cell type annotation provided in each dataset, we performed a global annotation using the mammary gland reference from Tabula Sapiens²⁹ and popV³⁰. Cells of hematopoietic lineage (B cells, CD4+ and CD8+ T cells, DCs, macrophages, NK and plasma cells) were removed from the primary tumors and from the lymph nodes, as well as from the controls. Tumor stromal cells, like endothelial cells (TEC), PVLs, and CAFs, and all other non-epithelial cells were also identified and removed. The filtered dataset contained essentially epithelial cells from normal and cancer breast tissues, together with those from the CTCs and from trophoblasts and early embryo (8K single cell samples). Notably, most of the mesenchymal-like CTCs were still retained (of breast origin), after removal of non-epithelial cells, apart from the pancreatic mesenchymal CTCs. Prostate CTCs were also retained as epithelial cells.

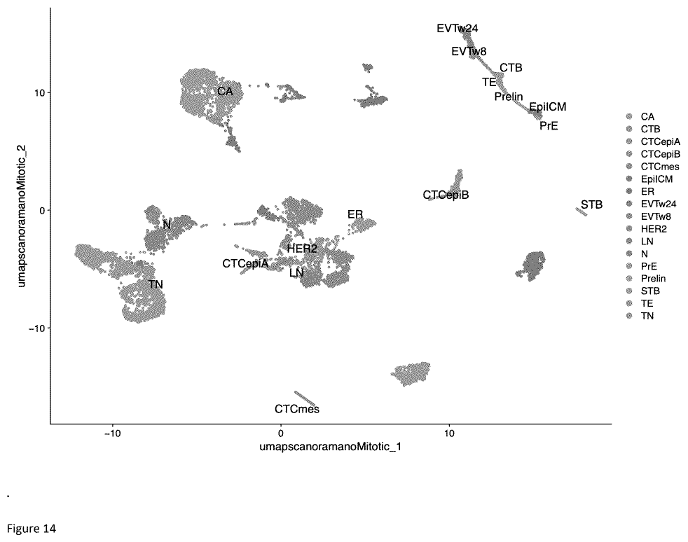

The UMAP plot of the integrated Scanorama dataset are shown in FIG. 14. Three major CTC-rich clusters (with epithelial A, with epithelial B, and mesenchymal CTCs), were identified. Notably, the epithelial A CTCs were clustered close to the HER2 breast cancer cells and metastatic breast cancer cells from lymph nodes (LN), while the epithelial B were located at the opposite side of the UMAP plot and close to the embryonic cell types. The mesenchymal CTCs are located on the bottom of the UMAP also distant from both epithelia CTC subgroups.

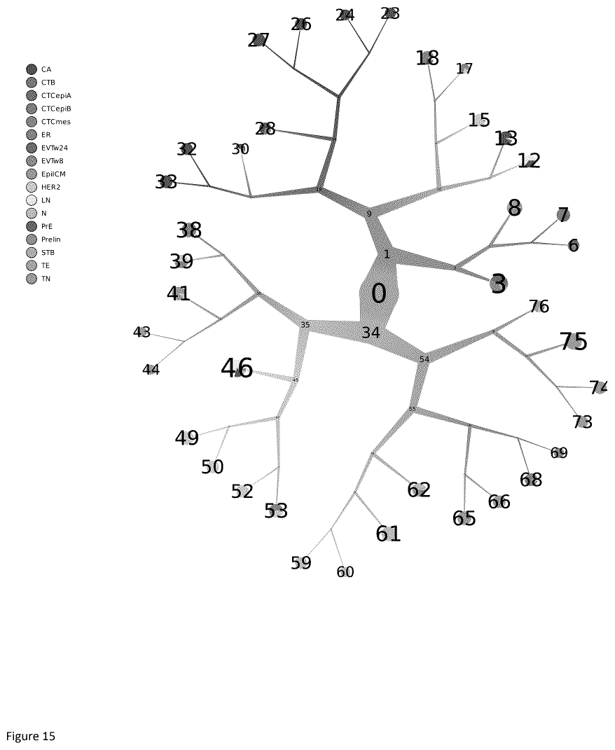

The integration of different single cell RNA profiles is still a developing field; therefore, we first investigated the inter-relations of the various samples and cell types using an approach orthogonal to the classic Louvain-based clustering algorithms. The plot in FIG. 15 shows the network obtained after shifted log normalization and divisive hierarchical spectral. This clustering representation showed that the RNA profiles of CTCs, trophoblast and embryo cells were strongly associated. Node 3 was the trophoblast node; nodes 6 and 7 were CTC nodes (93% and 100% respectively); node 8 was mostly of embryo origin (70%) with 6% of CTC and the remaining were from TNBC. Interestingly, almost all remaing CTCs (epithelial A and B) were located in two nodes (#28 and #30), within the metastatic cancer subnetwork (red nodes in the left and top hand side of of the plot.

### Automated Cell Annotation Confirms Functional Homology Between CTCS and Embryo Transcriptomes

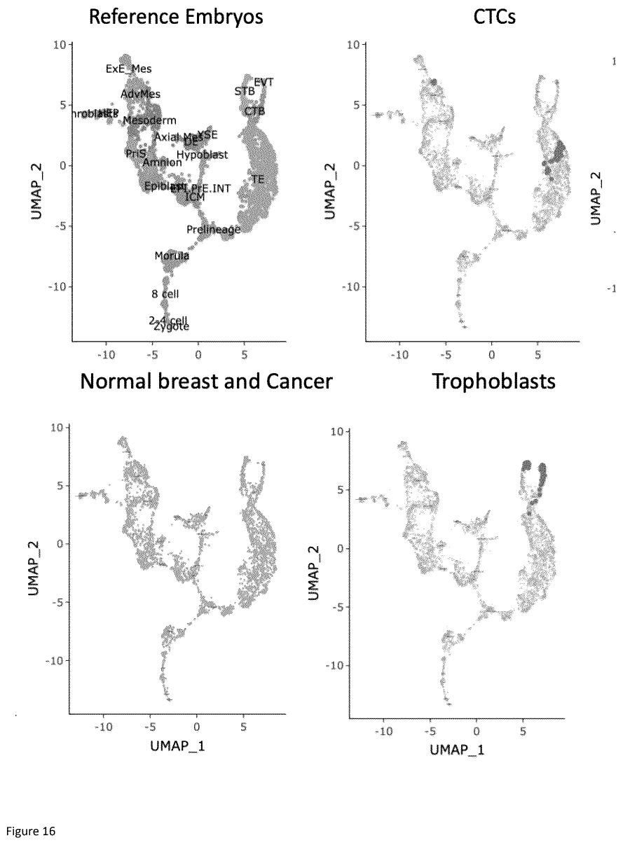

The results from the spectral clustering indicated again a relation between the RNA profiles of CTC epithelial B subgroup with trophoblast or early embryonic cell types. Could we confirm this relation using other computational tools? It cannot be stressed enough that the trophoblast has an invasive behavior as it infiltrates the mother's vascularization, during pregnancy, and at the same time it needs to counteract the maternal immune system. These two cellular skills should also belong to the CTCs, as prototypical seeding cancer cells, and would by themselves justify shared common transcriptional modules. To test this hypothesis, we looked for transcriptome similarities between CTCs and the different embryo cell types, by performing automated cell annotation; a procedure similar to using BLAST for the detection of homologous domains in oncoproteins. To this purpose, we used ShinyCell and multiple embryonic datasets³¹ as reference to map the CTCs onto the embryo developmental RNA map (FIG. 16). No CTCs were mapped on the trophoblast cell space (neither as EVT, CTB nor as STB cells). Unexpectedly, 72 CTCs (out of 551) were mapped as trophectoderm (TE), the tissue which originates trophoblasts. In a control test, no cells were called as TE from each of the non-metastatic cancer subtypes (400 samples each for TNBC, ER+ and HER2+ classes), nor from normal breast epithelial cells (0/703). There was only 1 TE cell called from the metastatic CA and LN cells, which was not significant, albeit might indicate an underlying biological match. Thus, the CTCs had a significant excess of cells annotated as trophectoderm (chi-square test, p-value<0.001) when compared with all other tumor or normal cells. The TE-like CTCs were mostly in the CTC epiB subgroup (64 out of 72, Fisher Test p-value<0.001), and were found in 50% of the CTC datasets (5 out of 10). We defined the functional modules shared between CTC and trophectoderm as CTC/trophectoderm modules.

### Identification of CTC/trophectoderm RNA Modules Shows That CTCS Are Related With Metastatic Cancer Cells

Then, we proceeded to identify all relevant CTC RNA modules, i.e., the one specific for all CTCs, the CTC subgroups, and those in common between CTC EpiB and the tropjhectoder layer. Among the up-regulated genes in common to all CTC subtypes, Two genes (ALDOA and PSMA6) were in common between the CTC, the early embryo cells with trophectoderm (but not the trophoblasts) and also the cancer cells derived from metastatic lymph nodes and their corresponding primary tumors. The primary, but not metastatic, tumors were also negative. Based on this finding we also looked for genes with this expression pattern, which could be associated with the metastatic process. We identified several RNA signatures specific for CTCs, or for CTC and embryonic tissues, or for CTC, embryonic tissues and metastatic primary cancers/lymph nodes.

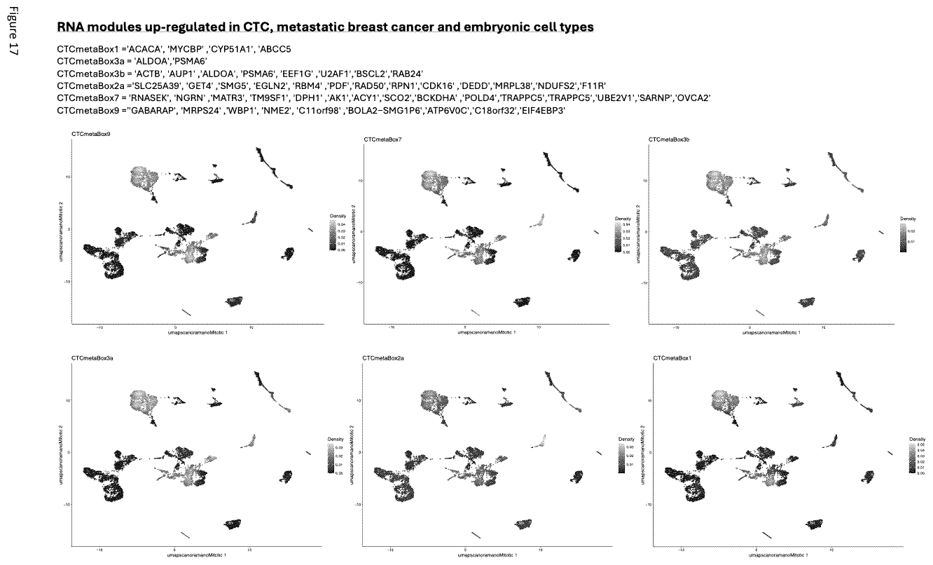

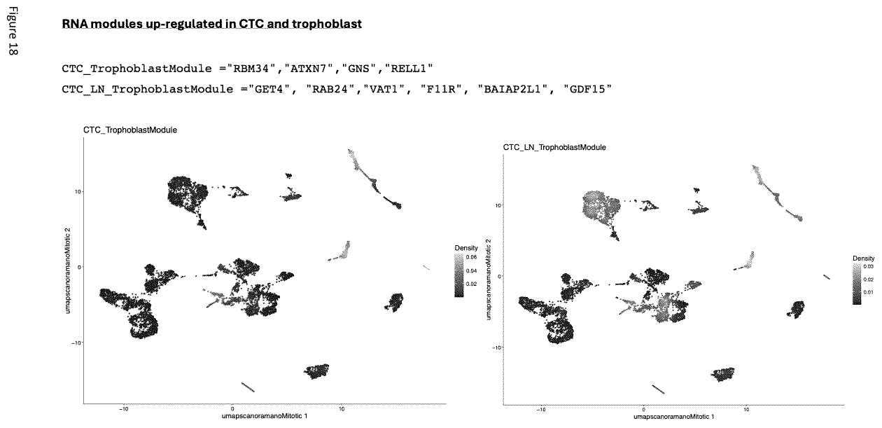

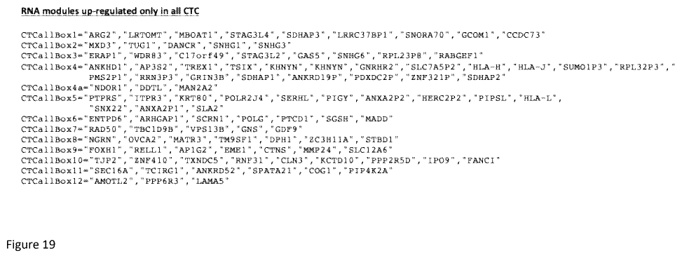

The UCell scores for the CTC RNA modules were computed and the results for the key CTC/trophectoderm modules are shown on the UMAP plots in FIGS. 17, 18, alongside the respective module compositions. For the pan-CTC RNA modules only the compositions are shown, in FIG. 19. Ucell is a gene signature scoring method based on the Mann-Whitney U statistic and its scores depend only on the relative gene expression in each individual cells.

### Negative Selection of PBMCs and Rare Large Blood Cells During CTC Enrichment

A common step in the purification of CTCs from blood of patients is the negative selection of blood cell. Usually an anti-CD45 (PTPRC) antibody is used to remove the excess of PBMCs from the CTC sample. Most of the CTC datasets available from GEO contained some PTPRC-positive cells but, perhaps surprisingly, the largest fraction of blood-derived and non-cancer cells was made of PTPRC-negative cells. Thus, we went on to identify markers which might be used in the removal of such residual contaminant PBMCs. These CTC contaminants were a large portion of CTC co-purified cells (about ⅔), but were rare in the whole blood: only 14 nucleated cells out of 10434 PBMCs, i.e. 0.13%, mapped to the “contaminant”Scanorama clusters.

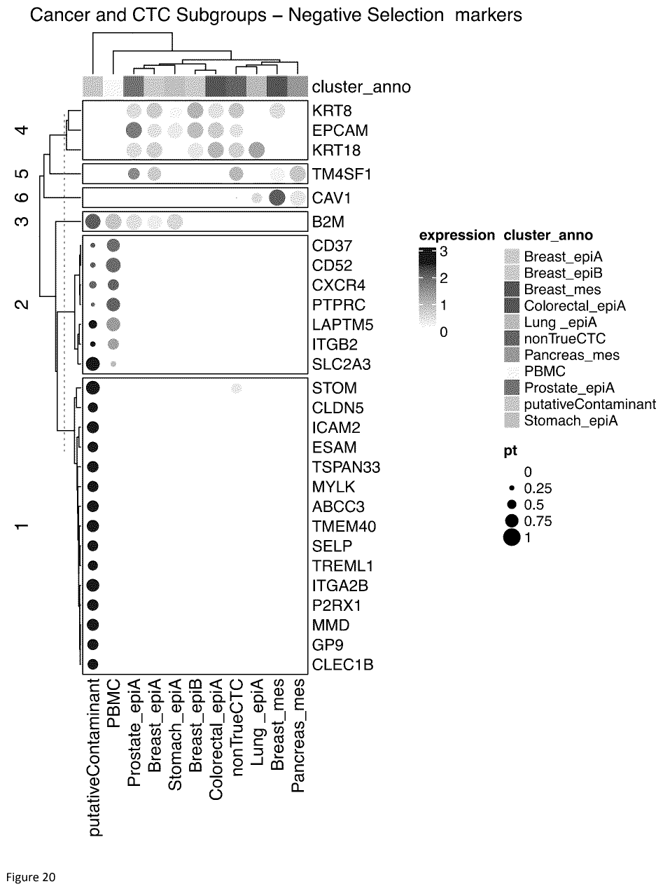

To identify robust CTCs markers, we performed nested cross-validation. First, we looked for the membrane markers to differentiate all the blood cells (PBMC and contaminants) from CTCs. These markers would be useful for the negative selection of blood, similarly to PTPRC/CD45. Six additional markers were identified, together with PTPRC/CD45: CXCR4, CD52, LAPTM5, CD37, ITGB2, and B2M. Finally, we looked specifically for markers differentiating the contaminant cells, mostly CD45-ve, from PBMC and CTCs. The model spanned a larger number of genes: SELP, GP9,TREML1, CLEC1B, MYLK, ITGA2B, MMD, TMEM40, STOM, TSPAN33, ESAM, P2RX1, SLC2A3, CLDN5, ABCC3, and ICAM2. These genes were not only expressed in platelets (or megakaryocytes), and in monocytes/macrophages, but also in endothelial cells, indicating a heterogeneous source of contaminants, likely large cells, possibly in complex with platelets or erythrocytes. The expression of these markers is shown, together with that of some controls, in FIG. 20. The genes expressed in PBMCs are those in the box no 2, while those for the removal of CD45-ve contaminants are in box no 1.

### Three Populations of Circulating Tumor Cells With Different Characteristics

We could separate a smaller mesenchymal CTC (VIM+/AXL1+) subpopulation from the larger portion of epithelial CTCs (EPCAM+). It was not possible to understand whether this was the representative ratio in vivo, since most of the CTCs were isolated using EPCAM, or other epithelial markers. Reassuringly, prostate CTCs were KLK2 positive and, conversely, all the KLK2+ cells in the integrated dataset were of prostate cancer origin.

We identified a few novel mesenchymal CTC markers, such as AXL and CAV1, that could be used to purify mesenchymal CTC, not only from breast cancer but also from pancreas adenocarcinomas. From our findings, we deduced that there is a need to perform agnostic and systematic studies of CTCs, using negative selection of blood cells, rather than positive selection with EPCAM (or other markers) which might restrict CTC yield to some cancer types or patients. For this purpose, we identified several targets for CTC purification by blood cells depletion, in particular from CD45-negative size selected cells. Antibodies for these markers can be used to enrich CTCs without using CTC-targeted antibodies and ensure an unbiased representation of all the different CTC states in the blood of a cancer patient. Additionally, negative selection of PBMCs would ensure proper identification of CTCs for cancers which are currently understudied. Notably, AXL1 and CAV emerged as two highly specific markers for currently understudied mesenchymal CTCs.

Furthermore, we were able to split epithelial CTCs into two divergent subgroups: one (epithelial A CTC) with high CD24/CDH1 expression and the second (epithelial B CTC) with high mitotic activity. The epithelial B CTCs had high and coordinated expression of several genes (e.g., LY6E, PRSS8 and CD276), which were also expressed in the trophoblast (placenta), among the normal tissues.

Importantly, CD276 (B7-H3), and not PDL1 (CD274), was the major immune-checkpoint gene expressed in CTCs. In non-malignant tissues, CD276 has a predominantly inhibitory role in adaptive immunity, suppressing T cell activation and proliferation. In malignant tissues, CD276 is an immune checkpoint molecule that inhibits tumor antigen-specific immune responses³². CD276/B7-H3 is the target of several anticancer agents such as enoblituzumab³³, and CAR T cells.³⁴ Some of the differentially expressed genes we detected in CTCs are also prominent in placenta, or trophoblast. Therefore, we investigated in depth any possible link between CTCs and trophoblast cells. There are three main trophoblast cell types: cytotrophoblast and syncytiotrophoblast that comprise the villous compartment and contribute to gas and nutrient exchange, and extra-villous trophoblast that invade and remodel the uterine wall and vessels, to supply maternal blood to the growing fetus.

### CTCs Share Genetic Programs With Both Trophectoderm and Metastatic Cancer Cells

Almost all the CTCs we collected are from breast, therefore we performed a comprehensive study of CTCs with normal and cancer tissues from breast, and of trophoblasts and early embryos. On the Scanorama integration the EpiB CTCs were located close to the embryo tissues. And when all the samples in the integrated dataset (over 8000 single cell profiles) were mapped onto a human embryo reference, only EpiB CTCs had a hit with embryonic cells, in particular with those from trophectoderm. Spectral clustering confrmed that the transcriptome profiles of CTCs were highly related with those of cells from trophoblasts and early embryos (including trophectoderm).

We identified several short signatures specific for the CTCs, or for the EpiA, EpiB, and mesenchymal CTC subtypes. Finally we identified a number of RNA modules which span CTCs and embryonic cells and also metastatic cancer cells.

In our study, we demonstrated for the first time a link between CTCs and trophectoderm cells. The concept of links between cancer and placenta development had been proposed earlier by several groups³⁵. Yet, as to now no confirmative work had been performed, nor any involved cellular, genetic, or molecular mechanism identified. We found here a gene network linking circulating tumor cells with embryonic cells, in particular of the highly indifferentiated trophectoderm. It is trophoectoderm from the early embryo that gives rise to the trophoblasts and in turn to placenta.

We showed here that CTCs hijack key modules in the genetic program of trophectoderm cells to become capable of first invading the vasculature and then attaining metastasis.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

### REFERENCES

1. Dillekås H, Rogers MS, Straume O. Are 90% of deaths from cancer caused by metastases? Cancer Med. 2019;8(12):5574-5576. doi:10.1002/cam4.2474
2. Brabletz T. To differentiate or not—routes towards metastasis. Nat Rev Cancer. 2012;12(6):425-436. doi:10.1038/nrc3265
3. Phan TG, Croucher PI. The dormant cancer cell life cycle. Nat Rev Cancer. 2020;20(7):398-411. doi:10.1038/s41568-020-0263-0
4. Guo B, Oliver TG. Partners in Crime: Neutrophil-CTC Collusion in Metastasis. Trends in Immunology. 2019;40(7):556-559. doi:10.1016/j. it.2019.04.009
5. Szczerba BM, Castro-Giner F, Vetter M, et al. Neutrophils escort circulating tumour cells to enable cell cycle progression. Nature. 2019;566(7745):553-557. doi:10.1038/s41586-019-0915-y
6. Diamantopoulou Z, Castro-Giner F, Schwab FD, et al. The metastatic spread of breast cancer accelerates during sleep. Nature. 2022;607(7917):156-162. doi:10.1038/s41586-022-04875-y
7. Vegliante R, Pastushenko I, Blanpain C. Deciphering functional tumor states at single-cell resolution. The EMBO Journal. 2022;41(2):e109221. Doi:10.15252/embj.2021109221
8. Trapnell C. Defining cell types and states with single-cell genomics. Genome Res. 2015;25(10):1491-1498. doi:10.1101/gr.190595.115
9. Rozenblatt-Rosen O, Regev A, Oberdoerffer P, et al. The Human Tumor Atlas Network: Charting Tumor Transitions across Space and Time at Single-Cell Resolution. Cell. 2020;181(2):236-249. doi:10.1016/j. cell.2020.03.053
10. Roth A, McPherson A, Laks E, et al. Clonal genotype and population structure inference from single-cell tumor sequencing. Nat Methods. 2016;13(7):573-576. doi:10.1038/nmeth.3867
11. Zhang AW, O'Flanagan C, Chavez EA, et al. Probabilistic cell-type assignment of single-cell RNA-seq for tumor microenvironment profiling. Nat Methods. 2019;16(10):1007-1015. doi:10.1038/s41592-019-0529-1
12. Gerlinger M, Rowan AJ, Horswell S, et al. Intratumor Heterogeneity and Branched Evolution Revealed by Multiregion Sequencing. New England Journal of Medicine. 2012;366(10):883-892. doi:10.1056/NEJMoa1113205
13. López S, Lim EL, Horswell S, et al. Interplay between whole genome doubling and the accumulation of deleterious alterations in cancer evolution. Nat Genet. 2020;52(3):283-293. doi:10.1038/s41588-020-0584-7
14. Steele CD, Abbasi A, Islam SMA, et al. Signatures of copy number alterations in human cancer. Nature. 2022;606(7916):984-991. doi:10.1038/s41586-022-04738-6
15. Yang J, Antin P, Berx G, et al. Guidelines and definitions for research on epithelial-mesenchymal transition. Nat Rev Mol Cell Biol. 2020;21(6):341-352. doi:10.1038/s41580-020-0237-9
16. Deshmukh AP, Vasaikar SV, Tomczak K, et al. Identification of EMT signaling cross-talk and gene regulatory networks by single-cell RNA sequencing. Proceedings of the National Academy of Sciences. 2021;118(19):e2102050118. doi:10.1073/pnas.2102050118
17. Vendramin R, Litchfield K, Swanton C. Cancer evolution: Darwin and beyond. The EMBO Journal. 2021;40(18): e108389. doi:10.15252/embj.2021108389
18. Liu SJ, Dang HX, Lim DA, Feng FY, Maher CA. Long noncoding RNAs in cancer metastasis. Nat Rev Cancer. 2021;21(7):446-460. doi:10.1038/s41568-021-00353-1
19. Clough E, Barrett T. The Gene Expression Omnibus database. Methods Mol Biol. 2016;1418:93-110. doi:10.1007/978-1-4939-3578-9_5
20. Schwartz GW, Zhou Y, Petrovic J, et al. TooManyCells identifies and visualizes relationships of single-cell clades. Nat Methods. 2020;17(4):405-413. doi:10.1038/s41592-020-0748-5
21. Lambert AW, Weinberg RA. Linking EMT programmes to normal and neoplastic epithelial stem cells. Nat Rev Cancer. 2021;21(5):325-338. doi:10.1038/s41568-021-00332-6
22. Bakir B, Chiarella AM, Pitarresi JR, Rustgi AK. EMT, MET, Plasticity, and Tumor Metastasis. Trends in Cell Biology. 2020;30(10):764-776. Doi:10.1016/j. Tcb.2020.07.003
23. Germain PL, Lun A, Meixide CG, Macnair W, Robinson MD. Doublet identification in single-cell sequencing data using scDblFinder. Published online May 16, 2022. doi:10.12688/f1000research.73600.2
24. Ahlmann-Eltze C, Huber W. Comparison of transformations for single-cell RNA-seq data. Nat Methods. 2023;20(5):665-672. doi:10.1038/s41592-023-01814-1
25. Luecken MD, Büttner M, Chaichoompu K, et al. Benchmarking atlas-level data integration in single-cell genomics. Nat Methods. 2022;19(1):41-50. doi:10.1038/s41592-021-01336-8
26. Nofech-Mozes I, Soave D, Awadalla P, Abelson S. Pan-cancer classification of single cells in the tumour microenvironment. Nat Commun. 2023;14(1):1615. doi:10.1038/s41467-023-37353-8
27. Duan B, Zhu C, Chuai G, et al. Learning for single-cell assignment. Sci Adv. 2020;6(44):eabd0855. doi:10.1126/sciadv. abd0855
28. Ding J, Adiconis X, Simmons SK, et al. Systematic comparison of single-cell and single-nucleus RNA-sequencing methods. Nat Biotechnol. 2020;38(6):737-746. doi:10.1038/s41587-020-0465-8
29. THE TABULA SAPIENS CONSORTIUM. The Tabula Sapiens: A multiple-organ, single-cell transcriptomic atlas of humans. Science. 2022;376(6594):eabl4896. doi:10.1126/science. abl4896
30. Ergen C, Xing G, Xu C, et al. Consensus prediction of cell type labels with popV. Published online August 21,2023:2023.08.18.553912. doi:10.1101/2023.08.18.553912
31. Zhao C, Reyes AP, Schell JP, et al. A Comprehensive Human Embryogenesis Reference Tool using Single-Cell RNA-Sequencing Data. Published online February 2, 2024:2021.05.07.442980. doi:10.1101/2021.05.07.442980
32. Kontos F, Michelakos T, Kurokawa T, et al. B7-H3: an attractive target for antibody-based immunotherapy. Clin Cancer Res. 2021;27(5):1227-1235. doi:10.1158/1078-0432.CCR-20-2584
33. Shenderov E, De Marzo AM, Lotan TL, et al. Neoadjuvant enoblituzumab in localized prostate cancer: a single-arm, phase 2 trial. Nat Med. 2023;29(4): 888-897. doi:10.1038/s41591-023-02284-w
34. Li D, Wang R, Liang T, et al. Camel nanobody-based B7-H3 CAR-T cells show high efficacy against large solid tumours. Nat Commun. 2023;14(1): 5920. doi: 10.1038/s41467-023-41631-w
35. Costanzo V, Bardelli A, Siena S, Abrignani S. Exploring the links between cancer and placenta development. Open Biology. 2018;8(6):180081. doi:10.1098/rsob.180081

1. A method for detection and subtyping of circulating tumor cells, the method comprising: at least isolating the circulating tumor cell, and determining the subtype.

2. The method of claim 1, wherein at least one key feature is a transcriptome, and/or a proteome.

3. The method of claim 2, wherein the activation of the CTC/trophectoderm modules indicates an increased likelihood of metastasis.

4. The method of claim 3, wherein the tumor is from breast or prostate or pancreas or lung or stomach or colon cancer.

5. A method for determining the likelihood of tumor metastases, the method comprising performing RNA sequencing on a biological sample from a subject to identify expressed genes in cancer cells of the CTC/trophectoderm modules; determining an expression composite score for each gene the CTC/trophectoderm modules; and using the composite score to assess the metastatic activity of the cells; wherein cells with higher CTC/trophectoderm module scores are more likely to metastasize.

6. The method of claim 5, wherein activation of CTC/trophectoderm modules induce a presence of circulating tumor cells (CTCs).

7. The method of claim 6, wherein the presence of CTCs with activation of CTC/trophectoderm modules indicates an increased likelihood of metastases.

8. The method of claim 7, wherein the tumor is from breast or prostate or pancreas or lung or stomach or colon cancer.

9. A method of treatment of a cancer, comprising determining the likelihood of tumor metastases according to claim 5, and administering to a subject in need thereof determined to have an increased likelihood of tumor metastasis an effective amount of a treatment for the cancer.

10. The method of claim 9, wherein the cancer is breast or prostate or pancreas or lung or stomach or colon cancer.
